(19) United States
(12) Patent Application Publication
(10) Pub. No.: US 2014/0228427 A1
(43) Pub. Date: Aug. 14, 2014

(54) PHARMACEUTICAL COMPOSITION CONTAINING WOGONIN AS AN ACTIVE INGREDIENT FOR PREVENTING OR TREATING ASTHMA

(71) Applicants: Sei Ryang Oh, Daejeon (KR); Kyung Seop Ahn, Daejeon (KR); Hyeong Kyu Lee, Daejeon (KR); Ha Young Jang, Daejeon (KR); Ok-Kyoung Kwon, Daejeon (KR); Jung Hee Kim, Daejeon (KR); Ji Won Park, Daejeon (KR); Doo-Young Kim, Daejeon (KR)

(72) Inventors: Sei Ryang Oh, Daejeon (KR); Kyung Seop Ahn, Daejeon (KR); Hyeong Kyu Lee, Daejeon (KR); Ha Young Jang, Daejeon (KR); Ok-Kyoung Kwon, Daejeon (KR); Jung Hee Kim, Daejeon (KR); Ji Won Park, Daejeon (KR); Doo-Young Kim, Daejeon (KR)

(73) Assignee: KOREA RESEARCH INSTITUTE OF BIOSCIENCE AND BIOTECHNOLOGY, Daejeon (KR)

(21) Appl. No.: 14/122,789

(22) PCT Filed: May 24, 2012
(86) PCT No.: PCT/KR2012/004139

§ 371 Date: Nov. 27, 2013

(30) Foreign Application Priority Data
May 27, 2011 (KR) 10-2011-0050736

(51) Int. Cl. A61K 31/352

(52) U.S. Cl. CPC A61K 31/352
USPC 514/456

(57) ABSTRACT

The present invention relates to a pharmaceutical composition for the prevention or treatment of asthma comprising Wogonin as an active ingredient. The Wogonin of the present invention is excellent not only in relieving airway hypersensitivity that causes asthma in vivo, in inhibiting reactive oxygen species generation in the airway, and in inhibiting the infiltration of inflammatory cells in the bronchus, but also in inhibiting the generation of IgE in serum and bronchoalveolar lavage fluid, and in inhibiting the expressions of Th2 cytokines in the lung. Therefore, the Wogonin of the present invention can be effectively used as a composition for the prevention, treatment, or improvement of asthma.

### BACKGROUND OF THE INVENTION

1. Field of the Invention

The present invention relates to a pharmaceutical composition for the prevention or treatment of asthma comprising Wogonin as an active ingredient.

2. Description of the Related Art

Asthma is the bronchial hypersensitivity caused by various stimulants coming in with breathing. Once stimulated, airway mucosa including bronchial mucosa swells up by inflammation, which makes the airway narrow so that cough with wheezing (breathing with high-pitched whistling sound) and convulsive dyspnea are developed.

Treatment agents for bronchial asthma are divided into a controller and a reliever. The controller is exemplified by inhaled glucocorticosteroids, anti-allergic drugs, and long acting bronchodilators, which are helpful to prevent the long term malignant progress of asthma. In the meantime, the reliever largely stands for long acting bronchodilators, which have been used for the relief of acute asthma exacerbation or bronchial infraction. They have no problems in relieving light asthma symptoms in any case, but have difficulty in treating intermediate stage or terminal stage asthma. In the case of using high dose of inhaled glucocorticosteroids, not only severe side effects but also inconvenience of use can be problems to patients.

Asthma typically requires long-term drug administration. However, the conventional asthma drugs such as steroids or smooth muscle relaxants have severe side effects such as immune dysfunction, infection, and cardiovascular risk, etc. Therefore, it is urgently required to develop a novel asthma treating agent having less side effects than the conventional drugs but better improvement or treatment effect especially on the basis of the fundamental approach to the disease.

Asthma treating agents nowadays such as long-acting or short-acting β2-agonists or inhaled corticosteroids, are only to relieve symptoms. So, it is required to develop a novel drug with less side effects based and more focused on the fundamental mechanism of asthma.

Recently, as an attempt to treat allergic disease fundamentally, immune regulators have been tried. The treatment with such immune regulators is characterized by activating Th1 immune response that is suppressed by excessive Th2 immunity frequently observed in allergy patients and by reducing IgE. For example, Xolair is a kind of asthma treating agents using anti-IgE antibody, which has been on the market since August 2003. In addition, CpG ODN (oligodeoxynucleotide), the specific bacteria DNA, is known to activate Th1 immune response actively, with which clinical test has been undergoing. Other new drug candidates using antibodies against various cytokines and chemokines generated because of imbalance of immune system have been tried.

Advair/Seretide of GSK, Singulair of Merk, Spririva of Boehringer Ingelheim, and Symbicort of AstraZeneca are so called blockbusters in the market of asthma/COPD treatment agents.

However, some of those treatment agents are facing inevitable sales reduction because of generic competition caused by patent expiry. Along with the growth of market, generic release and new drug lunch are expected to rush in.

10% of world population are suffering from asthma, one of and the most representative allergic inflammatory disease which is continuously growing according to the acceleration of environmental pollution. According to National Heart, Lung, and Blood Institute (USA) and European Respiratory Society, the annual cost for asthma care reaches $16.1 billion (US) or $16.3 billion (EU). Long-term administration is inevitable considering the characteristics of asthma. However, the conventional asthma drugs such as steroids or smooth muscle relaxants have severe side effects such as immune dysfunction, infection, and cardiovascular risk, etc. Therefore, it is urgently requested to develop a novel drug having improvement or more fundamental treatment effect on asthma with less side effects. Papers and patent documents have been reported to explain asthma treatment effect of plant originated natural extracts.

As an example of the conventional compositions for the prevention and treatment of asthma, Korean Patent Publication No. 2008-023570 describes a composition for asthma and allergic disease comprising the crude extract, polar solvent extract or nonpolar solvent extract of Salvia miltiorrhiza BUNGE as an active ingredient. Korean Patent Publication No. 2004-097820 also describes a composition for the prevention and treatment of asthma comprising alpha lipoic acid as an active ingredient. Korean Patent Publication No. 2010-062450 describes a composition for the prevention and treatment of allergy, inflammation, or asthma comprising Kluyveromyces marxianus isolated from Kefir grain. Even though many studies have been actively undergoing with focusing on various extracts, compounds, or strains to treat asthma, it has not been accomplished yet to develop a novel drug useful for the treatment of asthma.

In the course of study to develop a novel asthma treating agent using natural substances, the present inventors found out that the Wogonin compound which is one of flavonid compounds isolated from Scutellariae Radix, could reduce airway hypersensitivity in vivo, inhibit the generation of reactive oxygen species in the airway, and suppress the infiltration of inflammatory cells in the bronchus, so that it can be effectively used as a composition for the prevention, treatment, or improvement of asthma, leading to the completion of the present invention.

### SUMMARY OF THE INVENTION

It is an object of the present invention to provide a pharmaceutical composition for the prevention or treatment of asthma comprising Wogonin or a pharmaceutically acceptable salt thereof as an active ingredient.

It is another object of the present invention to provide a health food composition for the prevention or improvement of asthma comprising Wogonin or a pharmaceutically acceptable salt thereof as an active ingredient.

It is further an object of the present invention to provide a treatment method for asthma containing the step of administering a pharmaceutically effective dose of Wogonin or a pharmaceutically acceptable salt thereof to a subject having asthma.

In addition, it is also an object of the present invention to provide a use of Wogonin or a pharmaceutically acceptable salt thereof for the prevention or treatment of asthma.

To achieve the above objects, the present invention provides a pharmaceutical composition for the prevention or treatment of asthma comprising the Wogonin represented by the following formula 1 or a pharmaceutically acceptable salt thereof as an active ingredient:

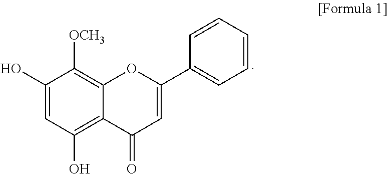 [SMILES: COc1c(O)cc(O)c2c(=O)cc(-c3ccccc3)oc12]

The present invention also provides a health food composition for the prevention or improvement of asthma comprising the Wogonin represented by formula 1 or a pharmaceutically acceptable salt thereof as an active ingredient.

The present invention further provides a treatment method for asthma containing the step of administering a pharmaceutically effective dose of the Wogonin represented by formula 1 or a pharmaceutically acceptable salt thereof to a subject having asthma.

In addition, the present invention provides a use of the Wogonin represented by formula 1 or a pharmaceutically acceptable salt thereof for the prevention or treatment of asthma.

### Advantageous Effect

As explained hereinbefore, the Wogonin of the present invention could reduce airway hypersensitivity which is a major reason of asthma in vivo, inhibit the generation of reactive oxygen species in the airway, suppress the infiltration of inflammatory cells in the bronchus, inhibit the generation of IgE in serum and bronchoalveolar lavage fluid, and suppress the expression of Th2 cytokine in the lung, so that it can be effectively used as a composition for the prevention, treatment, and improvement of asthma.

### BRIEF DESCRIPTION OF THE DRAWINGS

The application of the preferred embodiments of the present invention is best understood with reference to the accompanying drawings, wherein:

FIG. 1 is a graph illustrating the mouse weight changes according to the administration of the Wogonin of the present invention, investigated to confirm the inhibitory effect of the Wogonin on mouse weight loss.

FIG. 2 is a graph illustrating the Penh values over the administration of the Wogonin of the present invention, investigated to confirm the inhibitory effect of the Wogonin on airway hypersensitivity.

FIG. 3 is a graph illustrating the total IgE concentration in serum, measured to investigate whether or not the administered Wogonin of the present invention could reduce IgE concentration in serum.

FIG. 4 is a graph illustrating the total IgE concentration in bronchoalveolar lavage fluid, measured to investigate whether or not the administered Wogonin of the present invention could reduce IgE concentration in bronchoalveolar lavage fluid.

FIG. 5 is a graph illustrating the numbers of total inflammatory cells, eosinophils, and other inflammatory cells, measured to investigate the inhibitory effect of the administered Wogonin of the present invention on the population of inflammatory cells in bronchoalveolar lavage fluid.

FIG. 6 is a fluorescence diagram illustrating the expressions of IL-4, IL-13, MMP-9, and IL-6, measured to investigate the inhibitory effect of the administered Wogonin of the present invention on the expressions of Th2 cytokines in bronchoalveolar lavage fluid.

FIG. 7 is a graph illustrating the relative densities of IL-4, IL-6, IL-13, and MMP-9 to the normal control, measured to investigate the inhibitory effect of the administered Wogonin of the present invention on the expressions of Th2 cytokines in bronchoalveolar lavage fluid.

FIG. 8 is a graph illustrating the DCF fluorescent intensity to the normal control, measured to investigate the inhibitory effect of the administered Wogonin of the present invention on the generation of reactive oxygen species.

FIG. 9 is a photograph illustrating the inflammation extent around bronchiolus and blood vessels, observed to investigate the anti-inflammatory effect of the administered Wogonin of the present invention.

FIG. 10 is a graph illustrating the inflammation extent around bronchiolus and blood vessels, observed to investigate the anti-inflammatory effect of the administered Wogonin of the present invention in and around bronchiolus and blood vessels.

### DESCRIPTION OF THE PREFERRED EMBODIMENTS

Hereinafter, the present invention is described in detail.

The present invention provides a pharmaceutical composition for the prevention or treatment of asthma comprising the Wogonin represented by the following formula 1 or a pharmaceutically acceptable salt thereof as an active ingredient:

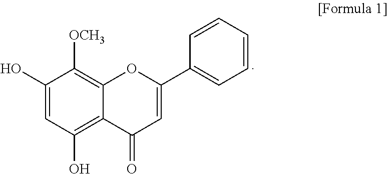 [SMILES: COc1c(O)cc(O)c2c(=O)cc(-c3ccccc3)oc12]

Particularly, the pharmaceutical composition of the present invention comprising the Wogonin represented by formula 1 or a pharmaceutically acceptable salt thereof can be effectively used as a pharmaceutical composition for the prevention or treatment of such diseases as bronchial asthma, allergic asthma, atopic asthma, non-atopic asthma, excise-induced asthma, aspirin-induced asthma, cardiac asthma, and alveolar asthma.

The said Wogonin in this invention can be extracted from Scutellariae Radix or prepared by the method well-known to those in the field of organic synthesis, or purchased in the market, but not always limited thereto. It is preferably separated and purified from Scultellariae Radix in this invention.

To investigate the effect of Wogonin on asthma, BALB/c mice sensitized with ovalbumin as an antigen was used as the airway inflammation animal model. BALB/c is the typical animal being used generally in the study of asthma breakout mechanism and screening of medicinal effect of a novel drug candidate and is also characterized by peculiar response against ovalbumin antigen (Hertz U, et al., Immunol Cell Biol, 74, 209-15, 1996). The mouse inflammation model is close to human bronchial asthma case in two aspects, that is they both exhibit eosinophilic inflammation in the lung and airway hypersensitivity. So, the drug dependent response in the mouse model has been studied in many ways (Dong-Keun Lee, Korean J. Pediatr., 46(10):952-957, 2003).

Unlike the control group, the asthma induced group (OVA), that was not administered with Wogonin, the inflammation mouse model administered with the Wogonin of Preparative Example 1 demonstrated significant inhibition of airway hypersensitivity, reduction of inflammatory cells in bronchoalveolar lavage fluid and decrease of IgE antibody level in serum. The expressions of Th2 cytokines including IL-4, IL-6, and IL-13 were also significantly inhibited in the inflammation mouse model administered with the Wogonin of Preparative Example 1. In addition, the generation of reactive oxygen species and the infiltration of inflammation cells in the airway mucosa were reduced in the group administered with the Wogonin of Preparative Example 1.

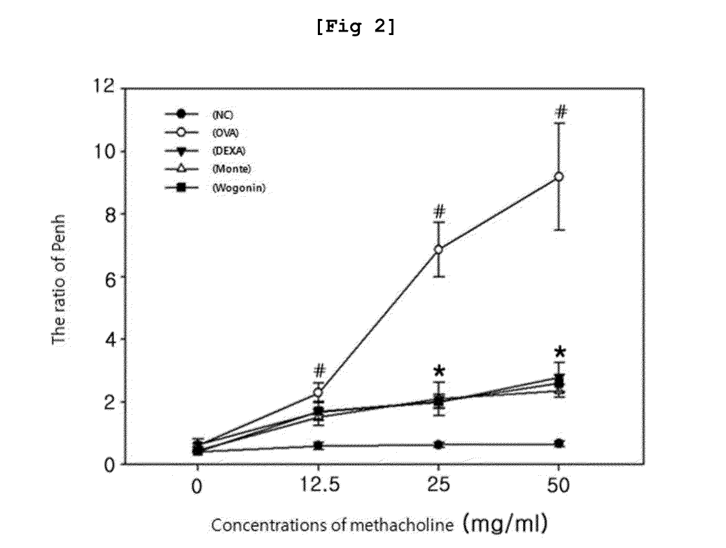

Airway hypersensitivity that can be an index used for the evaluation of the risk of asthma was measured by measuring lung capacity according to methacholine inhalation. Lung capacity was presented by Penh value over the increase of methacholine concentration. As a result, Penh value was rapidly increased in the asthma induced group (OVA), while Penh value was significantly decreased (1.7059-2.6055) in the group administered with the Wogonin of the present invention (see Table 1 and FIG. 2).

IgE plays an important role in bronchial asthma. Once sensitized with allergen, specific IgE antibody is formed and stays in blood or attached on the surface of mast cells or eosinophils. Particularly when exposed on the allergen again, this allergen is conjugated with IgE attached on mast cells to secret inflammatory mediators that cause airway inflammation and bronchoconstriction. In previous studies, it was confirmed that blood IgE level is closely related to the severeness of bronchial asthma (Noah T L, et al., Clin Immunol 2000; 97:43-49), suggesting the importance of IgE in the development of bronchial asthma.

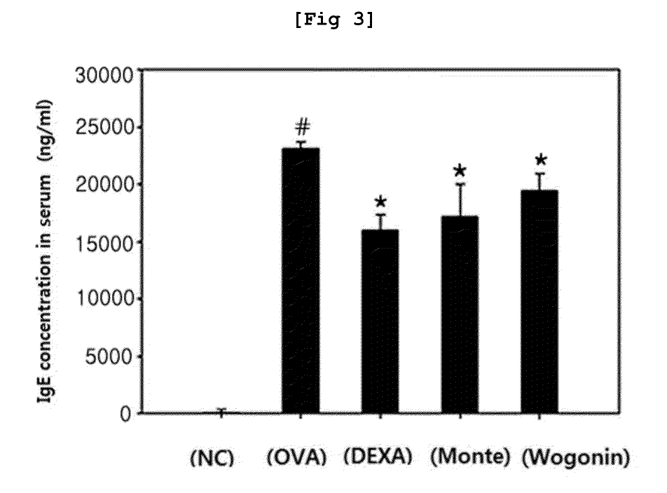

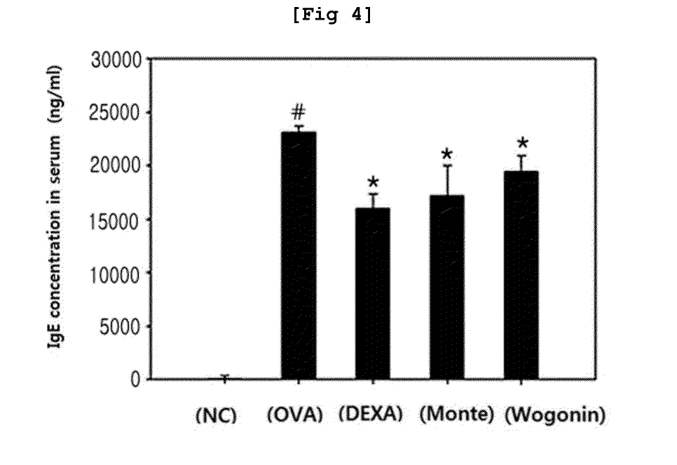

The present inventors measured the IgE antibody level in serum and in bronchoalveolar lavage fluid of the group administered with the Wogonin of the present invention. As a result, the IgE antibody concentration in serum was significantly reduced in the group administered with the Wogonin of the present invention (19418.52±1332.101 ng/ml), compared with that of the asthma induced group not treated with Wogonin (23139.51±484.45 ng/ml). In the meantime, the IgE antibody concentration in bronchoalveolar lavage fluid was significantly reduced in the group administered with the Wogonin of the present invention (12.10±1.87 ng/ml), compared with that of the asthma induced group not treated with Wogonin (19.02±2.43 ng/ml) (see Table 2, Table 3, FIG. 3, and FIG. 4).

In addition, airway inflammation was also investigated in asthma patients by performing bronchoalveolar lavage. As a result, it was confirmed that lymphocytes, mast cells, eosinophils, and activated macrophages were all increased in bronchoalveolar lavage fluid. Thus, asthma has been generally recognized as the airway inflammatory disease. It was reported that many of inflammatory cells were activated to secret various mediators to cause asthma. Therefore, the inhibition of inflammatory cells was considered to be related with the treatment of asthma (Haley K J, et al., Am J Respir Crit Care Med, 1998; 158:565-72).

The present inventors measured the number of inflammatory cells (macrophages, mast cells, eosinophils, and lymphocytes) in the group treated with the Wogonin of the present invention. As a result, the number of inflammatory cells was significantly reduced in the group treated with the Wogonin of the present invention (172.52±29.29), compared with that of the asthma induced group (524.30±43.22).

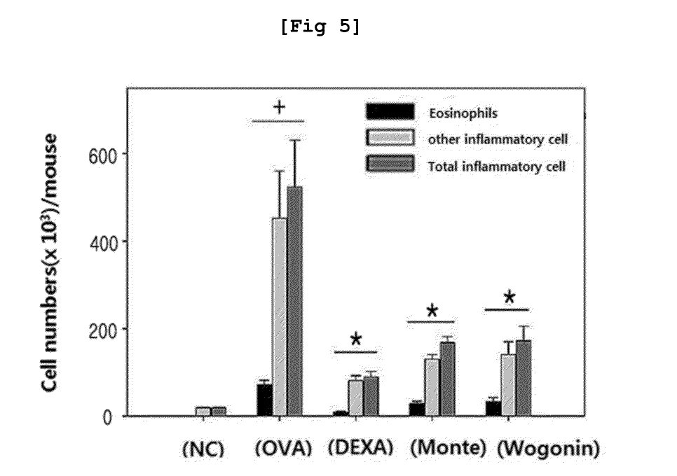

Among many inflammatory cells, eosinophils are the cells involved in cell mediated immunity by invading in tissues largely in the spleen and respiratory system. Eosinophils contain inflammatory proteins, so that they can damage directly airway epithelial cells, increase airway hypersensitivity, and induce mast cell degranulation, suggesting that they play an important role in the development of asthma. The number of eosinophils observed in the asthma induced group was 71.75±4.63, while the number of eosinophils in the group treated with the Wogonin of the present invention was reduced to 32.24±8.84 (see Table 4 and FIG. 5).

When an antigen is presented to Th2 cells by antigen presenting cells, Th2 cells are activated to secret Th2 cytokines such as IL-4, IL-6, and IL-13. These cytokines play a role in inducing allergic airway inflammation. Particularly, IL-4 and IL-13 stimulate B-cells to increase IgE production, while IL-6 amplifies the inflammation response (Wills-Karp M, et al., Science 1998; 282:2258-2261).

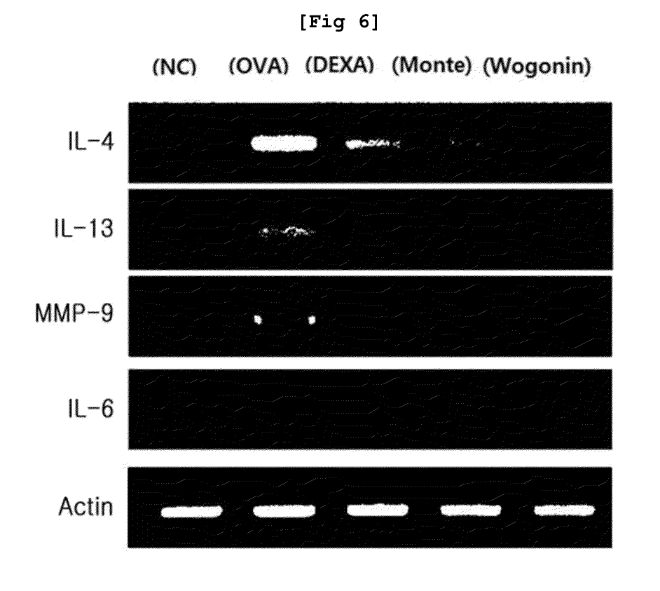

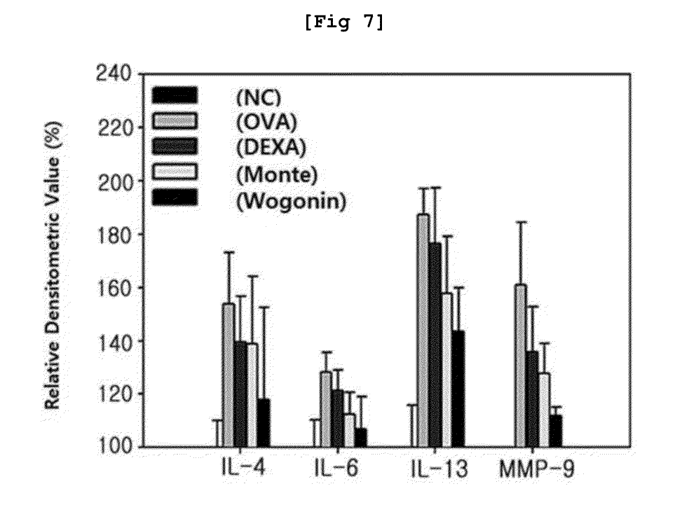

The present inventors investigated the expressions of cytokines in the lung of the group treated with the Wogonin of the present invention. As a result, the expressions of IL-4, IL-6, and IL-13 were suppressed by 26.14%, 16.64%, and 23.30% respectively in the group treated with the Wogonin of the present invention, compared with those of the asthma induced group. That is, the expressions of Th2 cytokines were suppressed in the asthma induced mouse by the treatment of the Wogonin of the present invention (see Table 5, FIG. 6, and FIG. 7).

Like in other inflammatory diseases, oxidative stress is increased in asthma. Reactive oxygen species is generated in macrophages and eosinophils. The increase of oxidative stress is closely related to the severity of disease. Oxidative stress increases inflammation response but decreases the response against steroids.

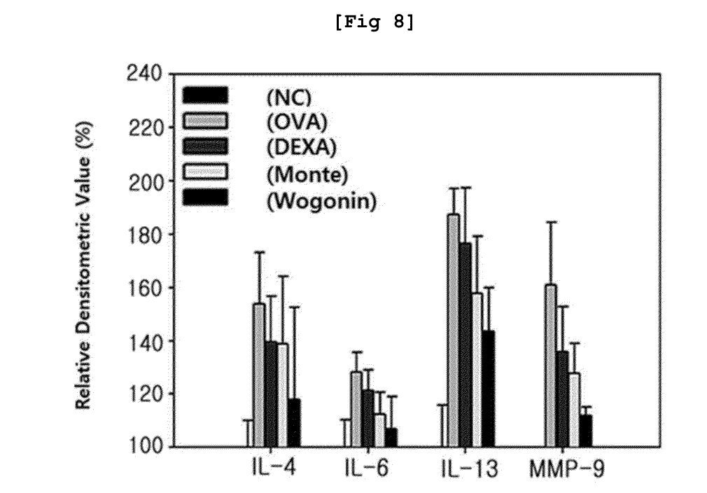

So, the present inventors investigated the inhibitory effect of the Wogonin of the present invention on the generation of reactive oxygen species in the group treated with the Wogonin of the present invention. As a result, the generation of reactive oxygen species was reduced by 68.42% in the group treated with the Wogonin of the present invention, compared with that of the asthma induced group (see Table 6 and FIG. 8).

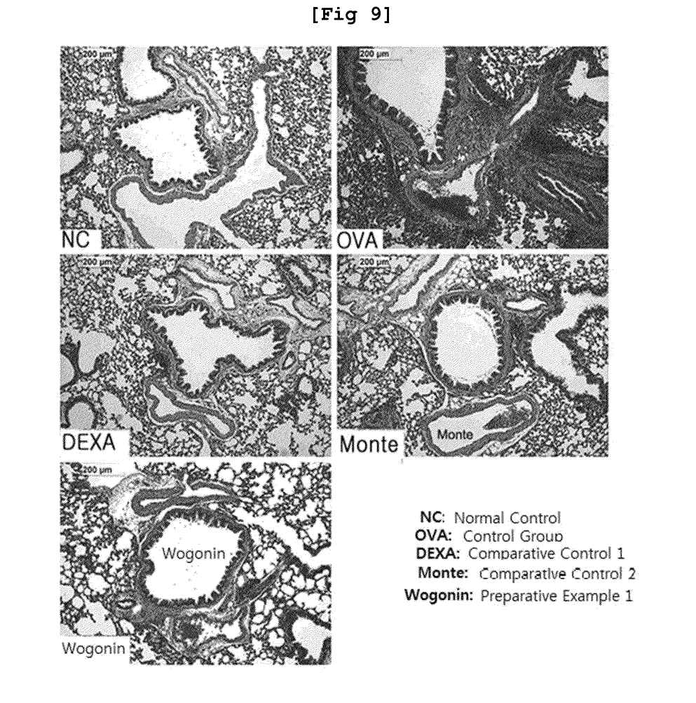

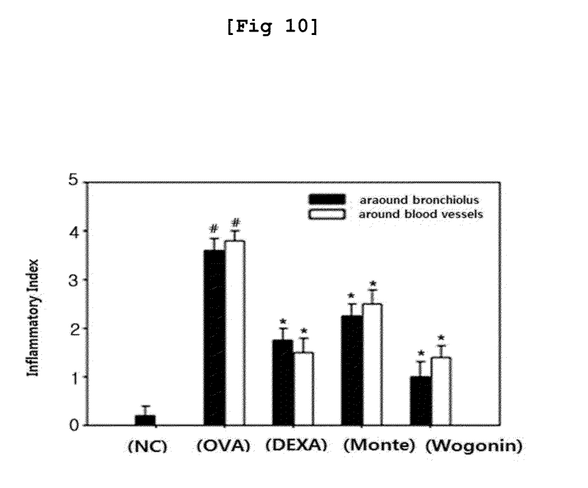

Once an antigen is introduced, the infiltration of inflammatory cells including eosinophils, neutrophils, and macrophages is peculiarly observed in the bronchus (Oh S W, et al., J. Immunol 2002; 168:1992-2000). Therefore, the present inventors evaluated the infiltration of inflammatory cells in the airway mucosa of the group treated with the Wogonin of the present invention. As a result, inflammation index around bronchiolus in the asthma induced group was confirmed to be 3.60±0.22, while it was 1.00±0.28 in the group treated with the Wogonin of the present invention, suggesting that inflammation index was significantly reduced in the group treated with the Wogonin of the present invention. In the meantime, inflammation index around blood vessel was significantly reduced in the group treated with the Wogonin of the present invention (1.40±0.22), compared with that of the asthma induced group (3.80±0.18) (see Table 7, FIG. 9, and FIG. 10).

From the above results, it was confirmed that the Wogonin of the present invention can be effectively used as a pharmaceutical composition for the prevention or treatment of asthma.

The present invention includes not only the Wogonin compound represented by formula 1 but also a pharmaceutically acceptable salt thereof, and a solvate, a hydrate, a recemate, or a stereoisomer possibly produced from the same.

The Wogonin compound represented by formula 1 of the present invention can be used as the form of a pharmaceutically acceptable salt, in which the salt is preferably acid addition salt formed by pharmaceutically acceptable free acids. The acid addition salt can be obtained from inorganic acids such as hydrochloric acid, nitric acid, phosphoric acid, sulfuric acid, hydrobromic acid, hydroiodic acid, nitrous acid and phosphorous acid, or non-toxic organic acids such as aliphatic mono/dicarboxylate, phenyl-substituted alkanoate, hydroxy alkanoate, alkandioate, aromatic acids and aliphatic/aromatic sulfonic acids. The pharmaceutically non-toxic salts are exemplified by sulfate, pyrosulfate, bisulfate, sulphite, bisulphite, nitrate, phosphate, monohydrogen phosphate, dihydrogen phosphate, metaphosphate, pyrophosphate, chloride, bromide, iodide, fluoride, acetate, propionate, decanoate, caprylate, acrylate, formate, isobutylate, caprate, heptanoate, propiolate, oxalate, malonate, succinate, suberate, cabacate, fumarate, maliate, butyne-1,4-dioate, hexane-1,6-dioate, benzoate, chlorobenzoate, methylbenzoate, dinitrobenzoate, hydroxybenzoate, methoxybenzoate, phthalate, terephthalate, benzenesulfonate, toluenesulfonate, chlorobenzenesulfonate, xylenesulfonate, phenylacetate, phenylpropionate, phenylbutylate, citrate, lactate, hydroxybutylate, glycolate, malate, tartrate, methanesulfonate, propanesulfonate, naphthalene-1-sulfonate, naphthalene-2-sulfonate and mandelate.

The acid addition salt of the present invention can be prepared by the conventional method known to those in the art. For example, the compound of formula 1 is dissolved in excessive acid aqueous solution and then the salt can be prepared by precipitation using a water-miscible organic solvent such as methanol, ethanol, acetone, or acetonitrile.

Equal amounts of the Wogonin compound of formula 1 and acid aqueous solution or alcohol are heated. Then, the solvent or the excessive acid is evaporated from the mixture, followed by drying the mixture to give addition salt or suction-filtering the precipitated salt to give the same.

A pharmaceutically acceptable metal salt can be prepared by using base. For example, alkali metal or alkali earth metal salt can be prepared by the following steps: dissolving the compound in excessive alkali metal hydroxide or alkali earth metal hydroxide solution, filtering non-soluble compound salt, evaporating the remaining solution, and drying thereof. At this time, the metal salt is preferably sodium salt, potassium salt, or calcium salt for the pharmaceutical purpose.

The composition comprising the Wogonin compound of the present invention can include the Wogonin at the concentration of 0.1-50 weight % by the total weight of the composition, but not always limited thereto.

The pharmaceutical composition of the present invention can additionally include generally used carriers, excipients and diluents.

The pharmaceutical composition of the present invention can be formulated for oral administration, for example powders, granules, tablets, capsules, suspensions, emulsions, syrups and aerosols, and for parenteral administration, for example external use, suppositories and sterile injections, etc. The carriers, excipients and diluents are exemplified by lactose, dextrose, sucrose, sorbitol, mannitol, xylitol, erythritol, maltitol, starch, acacia rubber, alginate, gelatin, calcium phosphate, calcium silcate, cellulose, methyl cellulose, microcrystalline cellulose, polyvinyl pyrrolidone, water, methylhydroxybenzoate, propylhydroxybenzoate, talc, magnesium stearate and mineral oil.

Formulations can be prepared by using generally used excipients or diluents such as fillers, extenders, binders, wetting agents, disintegrating agents and surfactant. Solid formulations for oral administration are tablets, pills, powders, granules and capsules. These solid formulations are prepared by mixing one or more suitable excipients such as starch, calcium carbonate, sucrose or lactose, gelatin, etc. Except for the simple excipients, lubricants, for example magnesium stearate, talc, etc, can be used. Liquid formulations for oral administrations are suspensions, solutions, emulsions and syrups, and the above-mentioned formulations can contain various excipients such as wetting agents, sweeteners, aromatics and preservatives in addition to generally used simple diluents such as water and liquid paraffin. Formulations for parenteral administration are sterilized aqueous solutions, water-insoluble excipients, suspensions, emulsions, lyophilized preparations, suppositories and injections. Water insoluble excipients and suspensions can contain, in addition to the active compound or compounds, propylene glycol, polyethylene glycol, vegetable oil like olive oil, injectable ester like ethylolate, etc. Suppositories can contain, in addition to the active compound or compounds, witepsol, macrogol, tween 61, cacao butter, laurin butter, glycerogelatin, etc.

The composition of the present invention can be administered orally or parenterally. The parenteral administration herein can be performed by systemic administration or local administration, but systemic administration is preferred and intravenous administration is more preferred.

The effective dosage of the composition of the present invention can be determined by those in the art according to weight and condition of patient, severity of disease, preparation of drug, and administration pathway and time. The effective dosage of the composition of the present invention is preferably 0.00010.03 g/kg per day, and more preferably 0.001-8 mg/kg per day. The administration frequency can be once a day or a few times a day. The above dosage cannot limit the scope of the present invention in any way.

The present invention also provides a health food composition for the prevention or improvement of asthma comprising the Wogonin represented by the following formula 1 or a pharmaceutically acceptable salt thereof as an active ingredient:

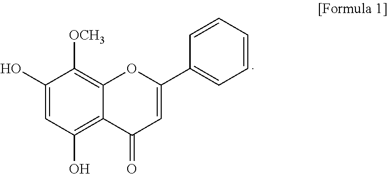 [SMILES: COc1c(O)cc(O)c2c(=O)cc(-c3ccccc3)oc12]

Particularly, the health food composition of the present invention comprising the Wogonin represented by formula 1 or a pharmaceutically acceptable salt thereof can be effectively used as a health food composition for the prevention or improvement of such diseases as bronchial asthma, allergic asthma, atopic asthma, non-atopic asthma, excise-induced asthma, aspirin-induced asthma, cardiac asthma, and alveolar asthma.

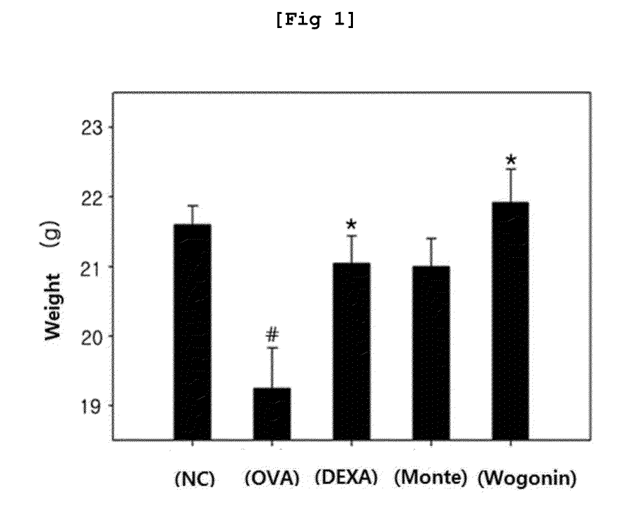

The Wogonin represented by formula 1 of the present invention could reduce airway hypersensitivity which is the major reason of asthma in vivo, inhibit the generation of reactive oxygen species in the airway, suppress the infiltration of inflammatory cells in the bronchus, inhibit the generation of IgE in serum and bronchoalveolar lavage fluid, and suppress the expression of Th2 cytokine in the lung, so that it can be effectively used as a health food composition for the prevention and improvement of asthma (see Tables 1˜7, and FIGS. 1˜10).

The Wogonin of the present invention can be added to health supplement foods such as foods and beverages for the prevention and improvement of asthma.

The food herein is not limited. For example, the Wogonin of the present invention can be added to meats, sausages, breads, chocolates, candies, snacks, cookies, pizza, ramyuns, flour products, gums, dairy products including ice cream, soups, beverages, tea, drinks, alcohol drinks and vitamin complex, etc, and in wide sense, almost every food applicable in the production of health food can be included.

The Wogonin compound represented by formula 1 of the present invention can be used as a food additive. In that case, the Wogonin compound represented by formula 1 of the present invention can be added as it is or as mixed with other food components according to the conventional method. The mixing ratio of active ingredients can be regulated according to the purpose of use (prevention or improvement). In general, to produce health food or beverages, the Wogonin compound represented by formula 1 of the present invention is added preferably by 0.1˜90 weight part to the total food weight. However, if long term administration is required for health and hygiene or regulating health condition, the content can be lower than the above but higher content can be accepted as well since the Wogonin compound represented by formula 1 of the present invention has been proved to be very safe.

The composition for health beverages of the present invention can additionally include various flavors or natural carbohydrates, etc, like other beverages. The natural carbohydrates above can be one of monosaccharides such as glucose and fructose, disaccharides such as maltose and sucrose, polysaccharides such as dextrin and cyclodextrin, and glucose alcohols such as xilytole, sorbitol and erythritol. Besides, natural sweetening agents (thaumatin, stevia extract, for example rebaudioside A, glycyrrhizin, etc.) and synthetic sweetening agents (saccharin, aspartame, etc.) can be included as a sweetening agent. The content of the natural carbohydrate is preferably 1˜20 g and more preferably 5˜12 g in 100 ml of the composition.

In addition to the ingredients mentioned above, the Wogonin compound of the present invention can include a variety of nutrients, vitamins, minerals (electrolytes), flavors including natural flavors and synthetic flavors, coloring agents and extenders (cheese, chocolate, etc.), pectic acid and its salts, alginic acid and its salts, organic acid, protective colloidal viscosifiers, pH regulators, stabilizers, antiseptics, glycerin, alcohols, carbonators which used to be added to soda, etc. The Wogonin compound of the present invention can also include natural fruit juice, fruit beverages and/or fruit flesh addable to vegetable beverages.

All the mentioned ingredients can be added singly or together. The mixing ratio of those ingredients does not matter in fact, but in general, each can be added by 0.1˜20 weight part per 100 weight part of the Wogonin compound isolated from Scutellariae Radix of the present invention.

The present invention further provides a treatment method for asthma containing the step of administering a pharmaceutically effective dose of the Wogonin represented by the following formula 1 or a pharmaceutically acceptable salt thereof to a subject having asthma:

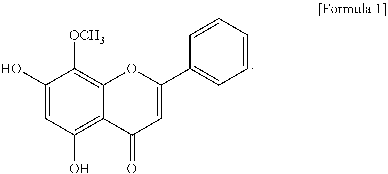 [SMILES: COc1c(O)cc(O)c2c(=O)cc(-c3ccccc3)oc12]

The term “pharmaceutically effective dose” herein indicates the amount enough to treat the disease with applicable, reasonable or risky concentration. The dose can be determined by considering many factors such as disease type, disease severity, drug activity, sensitivity to the drug, administration frequency and pathway, excretion, term of treatment, co-treatment drug and other factors regarded as relevant in the medicinal field.

The Wogonin of the present invention could reduce airway hypersensitivity which is the major reason of asthma in vivo, inhibit the generation of reactive oxygen species in the airway, suppress the infiltration of inflammatory cells in the bronchus, inhibit the generation of IgE in serum and bronchoalveolar lavage fluid, and suppress the expression of Th2 cytokine in the lung, so that it can be effectively used for the treatment of asthma.

The present invention also provides a use of the Wogonin represented by the following formula 1 or a pharmaceutically acceptable salt thereof for the prevention or treatment of asthma:

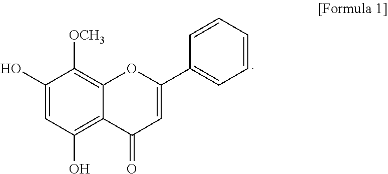 [SMILES: COc1c(O)cc(O)c2c(=O)cc(-c3ccccc3)oc12]

The Wogonin of the present invention could reduce airway hypersensitivity which is the major reason of asthma in vivo, inhibit the generation of reactive oxygen species in the airway, suppress the infiltration of inflammatory cells in the bronchus, inhibit the generation of IgE in serum and bronchoalveolar lavage fluid, and suppress the expression of Th2 cytokine in the lung, so that it can be effectively used as a composition for the prevention or treatment of asthma.

Practical and presently preferred embodiments of the present invention are illustrative as shown in the following Examples, Experimental Examples and Manufacturing Examples.

However, it will be appreciated that those skilled in the art, on consideration of this disclosure, may make modifications and improvements within the spirit and scope of the present invention.

### Preparative Example 1

### Preparation of Wogonin

Step 1: Preparation of Scutellariae Radix Extract

Dried Scutellariae Radix was minced. 30 L of methyl alcohol was added to 3.0 kg of the minced Scutellariae Radix, followed by extraction at room temperature. The extraction was repeated 4 times. As a result, 300 g of methyl alcohol extract was obtained.

Step 2: Preparation of Scutellariae Radix Extract Fraction

The methyl alcohol extract obtained in step 1 was dispersed in water, followed by fractionation using n-hexane and ethylacetate stepwise. As a result, 13.8 g of n-hexane fraction, 167 g of ethylacetate fraction, and 119.2 g of water fraction were obtained.

Step 3: Separation and Identification of Wogonin Compound

Silica gel column chromatography was performed with 160 g of the ethylacetate fraction obtained in step 2 using 30/1(v/v)˜5/1(v/v) of chloroform/methyl alcohol as a gradient elution solvent. Among the fractions obtained, those containing Wogonin were crystallized in methyl alcohol solvent. As a result, the compound (5.6 g) represented by formula 1 of the present invention was obtained. To analyze the structure of the compound, NMR and mass spectrometry were performed. As a result, the compound represented by formula 1 was identified to be Wogonin.

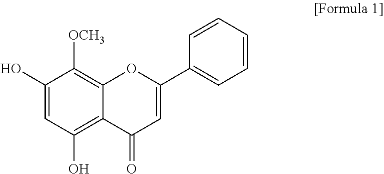 [SMILES: COc1c(O)cc(O)c2c(=O)cc(-c3ccccc3)oc12]

Compound Name: Wogonin

Molecular Formula: C15H10O5

Molecular Weight: 284.26

1H NMR (300 MHz, DMSO-d6) δ 6.98 (1H, s, H-3), 6.31 (1H, s, H-6), 8.07 (1H, m, H-2′), 7.60 (1H, m, H-3′), 7.60 (1H, m, H-4′), 7.60 (1H, m, H-5′), 8.07 (1H, m, H-3′), 3.89 (3H, s, OCH3).

13C NMR (300 MHz, DMSO-d6) δ 164.40 (C-2), 104.64 (C-3), 182.01 (C-4), 156.21 (C-5), 157.34 (C-7), 99.12 (C-6), 127.76 (C-8), 149.59 (C-9), 103.74 (C-10), 130.72 (C-1′), 126.36 (C-2′), 129.22 (C-3′), 131.46 (C-4′), 129.22 (C-5′), 126.36 (C-6′), 61.03 (C—OMe).

### Reference: Planta Medica-vol. 60, 493-494 (1994).

Test Animal and Bronchial Asthma Induction:

To investigate the effect of the Wogonin of the present invention on asthma, 6-week old female Balb/c mice in the weight of about 20 g were used as test animals. After spending one week for adaptation, basic physical examination was performed to select normal ones.

The test animal was sensitized with 200 μl of phosphate buffer (pH 7.4) containing 2 mg of aluminum hydroxide (A8222, Sigma, St. Louis, Mo.) and 20 μg of ovalbumin (A5503, Sigma, St. Louis, Mo.) by intraperitoneal injection at 2 weeks interval. 1% ovalbumin was inhaled in the mouse for 30 minutes by using an ultrasonic nebulizer from the 28^{th} day to the 30^{th} day after the first intraperitoneal injection of ovalbumin (OVA).

24 hours after the final antigen treatment, airway hypersensitivity was measured. 48 hours later, a lethal dose of pentobarbital (Entobar, Hanlim Pharm. Co. Ltd., Seoul, Korea) was administered to the mouse. Body weight was measured and tracheostomy was performed. Bronchoalveolar lavage was performed with 1.2 ml of saline, followed by sampling. The normal control group (NC) was administered with ovalbumin but not inhaled, the asthma induced group (OVA) was administered with ovalbumin and inhaled to induce bronchial asthma, the comparative group 1 (DEXA) was orally administered with dexamethasone (3 mg/kg, PO: D4902, Sigma, St. Louis, Mo.) one hour before inhaling ovalbumin, the comparative group 2 (Monte) was orally administered with montelukast (30 mg/kg, PO) one hour before inhaling ovalbumin, and the experimental group (Wogonin) was orally administered with the Wogonin of Example 1 (30 mg/kg, PO) one hour before inhaling ovalbumin. Each group was composed of 5 white mice.

The average body weight of the normal control group was 21.60±0.25 g, while the average body weight of the asthma induced group was 19.72±0.53 g, suggesting that the asthma induced group demonstrated significant weight loss. In the meantime, the average body weight of the dexamethasone administered group was 21.04±0.35 g, the average body weight of the montelukast administered group was 21.08±0.36 g, and the average body weight of the Wogonin administered group was 21.92±0.43 g, suggesting that the average body weights of these groups were recovered near to the average body weight of the normal control group (FIG. 1).

Statistical Analysis

All the measured values were presented as mean±error. Mean value and standard error (mean±S.E.) were calculated according to various variants. Comparison among the groups was performed by Mann-whitney U test using SPSS 10.0. It was judged as statistically significant when p value was less than 0.05.

### Example 1

### Measurement of Airway Hypersensitivity

The effect of the Wogonin of the present invention on asthma related airway hypersensitivity was evaluated by measuring enhanced pause (Penh) which presents the grade of airway obstruction calculated by measuring airway resistance using one chamber plethysmorgraphy (All Medicus, Seoul). For the measurement of Penh, base value was measured under normal breathing condition and then PBS was inhaled by using an ultrasonic nebulizer for 3 minutes, followed by measuring for 3 minutes. Then, methacholine (A2251, Sigma, St. Louis, Mo.), the histamine, which is generally used to diagnose bronchial asthma, was inhaled to the animal with raising the concentration gradually (12, 25, and 50 mg/ml), followed by measuring Penh. The ratio of Penh increased over the concentrations of methacholine inhaled was presented as % and baseline Penh (saline challenge) was presented as 100%. The results are shown in Table 1 and FIG. 2.

TABLE 1

| | Penh value | | | |
|---|---|---|---|---|
| | 0 mg/ml | 12.5 mg/ml | 25 mg/ml | 50 mg/ml |
| Preparative | 0.4261 ± 0.0069 | 1.7059 ± 0.3028 | 2.0166 ± 0.2248 | 2.6055 ± 0.2280 |
| Example 1 | | | | |
| (Wogonin) | | | | |
| Normal | 0.4206 ± 0.0217 | 0.5970 ± 0.1024 | 0.6336 ± 0.0682 | 0.6639 ± 0.0682 |
| Control | | | | |
| Group (NC) | | | | |
| Control | 0.6299 ± 0.0617 | 2.2917 ± 0.3070 | 6.8642 ± 0.8313 | 9.1854 ± 1.6230 |
| Group (OVA) | | | | |
| Comparative | 0.6486 ± 0.1526 | 1.6750 ± 0.0820 | 1.9963 ± 0.1577 | 2.7864 ± 0.4293 |
| Group 1 | | | | |
| (DEXA) | | | | |
| Comparative | 0.4525 ± 0.0313 | 1.5249 ± 0.2432 | 2.1006 ± 0.4758 | 2.3576 ± 0.1741 |
| Group 2 | | | | |
| (Monte) | | | | |

As shown in Table 1 and FIG. 2, Penh value was increased gradually according to the increase of the concentration of methacholine in the normal control group, while Penh value was increased rapidly and significantly in the asthma induced group.

In the meantime, Penh value was significantly decreased regardless of methacholine concentration in the comparative control groups 1 and 2 and the drug administered groups (dexamethasone administered group, montelukast administered group, and Wogonin administered group) than in the asthma induced group. This difference was more apparent when high dose of methacholine was inhaled.

Therefore, it was confirmed that the Wogonin of the present invention inhibits airway hypersensitivity, the cause of asthma, so that it can be effectively used for the treatment of asthma.

### Example 2

### Measurement of IgE in Serum and bronchoalveolar Lavage Fluid

To investigate the effect of the Wogonin of the present invention on asthma, the inventors measured IgE concentration in serum and bronchoalveolar lavage fluid which is related to the severity of asthma by enzyme immunoassay.

Serum and bronchoalveolar lavage fluid taken from each group were loaded in 90-well plate on which 0.1M NaHCO₃ buffer (pH 8.3) containing 20 μg/ml of ovalbumin (OVA) was coated at 4° C. for overnight. Non-specific reaction was blocked by PBS containing 1% bovine serum albumin. Serum sample was diluted at the ratio of 1:400, followed by reaction at room temperature for 2 hours.

After washing the plate well, the sample was reacted with anti-mouse IgE monoclonal antibody diluted at the ratio of 1:300 for 2 hours. HRP-conjugated goat anti-rat IgG polyclonal A was diluted at the ratio of 1:4000, which was added thereto for further reaction at room temperature for 1 hour, followed by washing.

For color development, 3.3′5.5′-tetramethylbenzidine substrate was added, followed by measuring OD₆₅₀. The results of analyzing IgE concentration in serum were shown in Table 2 and FIG. 3. The results of analyzing IgE concentration in bronchoalveolar lavage fluid were shown in Table 3 and FIG. 4.

TABLE 2

| | Total IgE Conc. in Serum |
|---|---|
| | (ng/ml) |
| Preparative Example 1 | 19418.52 ± 1332.101 |
| (Wogonin) | |
| Normal Control (NC) | 90.12 ± 239.06 |
| Control Group (OVA) | 23139.51 ± 484.45 |
| Comparative Control 1 | 15991.36 ± 1194.92 |
| (DEXA) | |
| Comparative Control 2 | 17176.54 ± 2542.98 |
| (Monte) | |

As shown in Table 2 and FIG. 3, it was confirmed that the IgE antibody concentration in serum was significantly reduced in the group administered with the Wogonin of the present invention (Preparative Example 1) (19418.52±1332.101 ng/ml), compared with that of the asthma induced group (OVA) (23139.51±484.45 ng/ml).

TABLE 3

| | Total IgE Conc. in |
|---|---|
| | bronchoalveolar lavage |
| | fluid (ng/ml) |
| Preparative Example 1 | 12.10 ± 1.87 |
| (Wogonin) | |
| Normal Control (NC) | 4.93 ± 1.04 |
| Control Group (OVA) | 19.02 ± 2.43 |
| Comparative Control 1 | 10.15 ± 1.38 |
| (DEXA) | |
| Comparative Control 2 | 10.15 ± 1.58 |
| (Monte) | |

As shown in Table 3 and FIG. 4, the concentration of IgE antibody in bronchoalveolar lavage fluid was not significantly changed but rather decreased in the group administered with the Wogonin of the present invention (12.10±1.87 ng/ml), compared with that of the asthma induced group (19.02±2.43 ng/ml).

Therefore, it was confirmed that the Wogonin of the present invention inhibited the IgE concentration in relation to asthma, so that it could be effectively used for the treatment of asthma.

### Example 3

### Analysis of Inflammatory Cells in Bronchoalveolar Lavage Fluid

To investigate the effect of the Wogonin of the present invention on asthma, the present inventors measured the increase or decrease of inflammatory cell number which is believed to be involved in asthma.

As soon as bronchoalveolar lavage fluid was recovered from each group, it was stained with Trypan blue. Then, the number of cells, except dead cells, was counted by hemocytometer. The sample was smeared with Cytospin II, followed by Diff-Quick staining (Sysmex, Switzerland) to calculate the numbers of eosinophils and other inflammatory cells. The results are shown in Table 4 and FIG. 5.

TABLE 4

| | Cell Number | | |
|---|---|---|---|
| | (×10³ cell/mouse) | | |
| | eosinophils | other cells | total cells |
| Preparative | 32.24 ± 8.84 | 140.28 ± 26.24 | 172.52 ± 29.29 |
| Example 1 | | | |
| (Wogonin) | | | |
| Normal Control | 0.00 ± 0.00 | 19.24 ± 1.0 | 19.24 ± 1.22 |
| (NC) | | | |
| Control Group | 71.75 ± 4.63 | 452.55 ± 93.48 | 524.30 ± 43.22 |
| (OVA) | | | |
| Comparative | 8.24 ± 1.31 | 81.00 ± 10.40 | 89.24 ± 6.98 |
| Control 1 | | | |
| (DEXA) | | | |
| Comparative | 27.88 ± 2.80 | 130.12 ± 9.22 | 167.68 ± 8.80 |
| Control 2 | | | |
| (Monte) | | | |

As shown in Table 4 and FIG. 5, the number of total inflammatory cells was 19.24±1.22 in the normal control group, 524.30±43.22 in the asthma induced group, 89.24±6.98 in the dexamethasone administered group (comparative control 1), 167.68±8.80 in the montelukast administered group (comparative control 2), and 172.52±29.29 in the group administered with the Wogonin of the present invention (Preparative Example 1), suggesting that the number of total inflammatory cells was all reduced in the drug treated groups, compared with that of the asthma induced group.

The number of other inflammatory cells was significantly reduced (140.28±26.24).

Particularly, the number of eosinophils which are the cells largely involved in cell-mediated immunity by invading in tissues and are spread throughout cells, spleen, and respiratory system with inflammatory protein to play an important role in the progress of asthma, was respectively 0.00±0.00 in the normal control, 71.75±4.63 in the asthma induced group, 8.24±1.31 in the dexamethasone administered group (comparative control 1), 27.88±2.80 in the montelukast administered group (comparative control 2), and 32.24±8.84 in the group administered with the Wogonin of the present invention (Preparative Example 1), suggesting that the number of eosinophils was significantly reduced in all the drug treated groups, compared with that of the asthma induced group.

Therefore, it was confirmed that the Wogonin of the present invention not only reduced the population of inflammatory cells in relation to asthma but also reduced the number of eosinophils affecting asthma, so that it could be effectively used for the treatment of asthma.

### Example 4

### Analysis of Cytokine mRNA

To investigate the effect of the Wogonin of the present invention on asthma, asthma related cytokine mRNA was measured.

RNA was isolated from the lung by using trizol (Invitrogen) in order to measure the expressions of Th2 cytokines such as IL-4, IL-6, and IL-13 in the supernatant of bronchoalveolar lavage fluid taken from each group, followed by reverse transcriptase polymerase chain reaction (quantitative RT-PCR, Promega, Wis.). When the lowest expression was indicated as 1, relative densitometric value (RDV) was produced by that to evaluate the expression inhibition effect. The results are shown in Table 5 and FIG. 6 and FIG. 7.

TABLE 5

| | Relative Densitometric Value, RDV) (%) | | |
|---|---|---|---|
| | (NC:NC = 100%) | | |
| | IL-4 | IL-6 | IL-13 |
| Preparative | 118.11 ± 29.80 | 106.87 ± 10.47 | 143.68 ± 14.08 |
| Example 1 | | | |
| (Wogonin) | | | |
| Normal | 100.00 ± 7.86 | 100.00 ± 9.02 | 100.00 ± 12.28 |
| Control (NC) | | | |
| Control Group | 159.91 ± 14.87 | 128.20 ± 6.51 | 187.35 ± 7.49 |
| (OVA) | | | |
| Comparative | 139.60 ± 13.34 | 121.37 ± 6.78 | 176.55 ± 16.13 |
| Control 1 | | | |
| (DEXA) | | | |
| Comparative | 138.86 ± 19.52 | 112.39 ± 7.16 | 157.67 ± 16.60 |
| Control 2 | | | |
| (Monte) | | | |

As shown in Table 5, the expressions of Th2 cytokines such as IL-4, IL-6, and IL-13 in the supernatant of bronchoalveolar lavage fluid were reduced in all the drug treated groups including the group administered with the Wogonin of the present invention, compared with those of the asthma induced group. Particularly, the inhibition effect was highest in the group treated with the Wogonin of Preparative Example 1 of the present invention.

Therefore, it was confirmed that the Wogonin of the present invention reduced the expressions of Th2 cytokines such as IL-4, IL-6, and IL-13 in relation to asthma, so that it could be effectively used for the treatment of asthma.

### Example 5

### Measurement of Reactive Oxygen Species Generation

The increase of oxidative stress results in the decrease of reaction to steroids. To investigate the effect of the Wogonin of the present invention on asthma, the present inventors measured the generation of reactive oxygen species which is apparent in asthma like in other inflammatory diseases.

A portion of bronchoalveolar lavage fluid of each group was washed with PBS, to which 10 μM of 2,7-Dichlorofluorescein diacetate (DCFH-DA, 35845, Sigma, St. Louis, Mo.), one of the fluorescent reagents, was added. The mixture stood at room temperature for 10 minutes in the darkness, and then measured fluorescence with a spectrofluorometer (Ex=480 nm Em=522 nm). The results are presented as % by the value of the normal control, which are shown in Table 6 and FIG. 8.

TABLE 6

| | Reactive oxygen species |
|---|---|
| | generation |
| | DCF fluorescence |
| | (NC:NC = 100%) |
| Preparative Example 1 | 60.91 ± 4.14 |
| (Wogonin) | |
| Normal Control (NC) | 100.33 ± 4.76 |
| Control Group (OVA) | 192.89 ± 27.36 |
| Comparative Control 1 | 116.55 ± 9.28 |
| (DEXA) | |
| Comparative Control 2 | 104.48 ± 9.80 |
| (Monte) | |

As shown in Table 6, the amount of generated reactive oxygen species in the group administered with the Wogonin of Preparative Example 1 of the present invention was reduced 68.42% by that of the asthma induced group, which presented higher inhibition effect than those of comparative control 1 (dexamethasone; 39.58%) and comparative control 2 (montelukast; 45.84%).

Therefore, it was confirmed that the Wogonin of the present invention was excellent in reducing the generation of reactive oxygen species generally observed in asthma cases, so that it could be effectively used for the treatment of asthma.

### Example 6

### Histopathological Test

The infiltration of inflammatory cells including eosinophils, neutrophils, and macrophages is observed in the bronchus sensitized with an antigen. To investigate the effect of the Wogonin of the present invention on asthma, the inventors performed histopathological test with the lung without bronchoalveolar lavage.

The lung extracted without bronchoalveolar lavage from each group was fixed with formalin and embedded in paraffin. Then, 4 μm thick permanent tissue sections were prepared, followed by H&E staining. Upon completion of H&E staining, inflammatory index was measured from 5 random regions of each section, and then mean value was calculated.

The inflammatory index 0 indicates that no inflammatory cells were observed around bronchus, and the inflammatory index 1 indicates that some inflammatory cells were observed intermittently. The inflammatory index 2 indicates that one, two or three thin inflammatory cell layers were observed in most parts of bronchus. The inflammatory index 3 indicates that two to three or up to five inflammatory cell layers were observed in most parts of bronchus, and the inflammatory index 4 indicates that at least 5 thick inflammatory cell layers were observed in most parts of bronchus. The results are shown in Table 7, FIG. 9 and FIG. 10.

TABLE 7

Inflammatory Index

| | around | around blood |
|---|---|---|
| | bronchiolus | vessels |
| Preparative Example 1 | 1.00 ± 0.28 | 1.40 ± 0.22 |
| (Wogonin) | | |
| Normal Control | 0.00 ± 0.00 | 0.00 ± 0.00 |
| (NC) | | |
| Control Group | 3.60 ± 0.22 | 3.80 ± 0.18 |
| (OVA) | | |
| Comparative Control 1 | 1.75 ± 0.19 | 1.50 ± 0.22 |
| (DEXA) | | |
| Comparative Control 2 | 2.25 ± 0.22 | 2.50 ± 0.25 |
| (Monte) | | |

In the asthma induced group, numbers of inflammatory cells including eosinophils were infiltrated around bronchiolus and over-proliferated epithelial cells and thickened tracheal smooth muscle were also observed.

In the meantime, as shown in Table 7, the infiltration of inflammatory cells was significantly inhibited in the drug administered groups. Particularly, the inflammatory index around bronchiolus was 3.60±0.22 in the asthma induced group, while it was 1.75±0.19 in the comparative control group 1 (DEXA), 2.25±0.22 in the comparative control group 2 (Monte), and 1.00±0.28 in the group administered with the Wogonin of Preparative Example 1 of the present invention, indicating that the inflammatory indexes in those drug treated groups were all significantly reduced compared with that of the asthma induced group.

The inflammatory index around blood vessels was 3.80±0.18 in the asthma induced group, while the inflammatory index was 1.50±0.22 in the comparative control group 1, and 0.50±0.25 in the comparative control group 2, and 1.40±0.22 in the group treated with the Wogonin of Preparative Example 1 of the present invention, indicating that the inflammatory indexes in those drug treated groups were significantly reduced, compared with that of the asthma induced group.

Therefore, it was confirmed that the Wogonin of the present invention was excellent in inhibiting inflammatory cells including eosinophils, neutrophils, and macrophages in the bronchus sensitized with an antigen, so that it can be effectively used for the treatment of asthma.

From the above results of Examples 1-6, it was confirmed that the Wogonin of the present invention reduced airway hypersensitivity which is the major reason of asthma in vivo, inhibited the generation of reactive oxygen species in the airway, suppressed the infiltration of inflammatory cells in the bronchus, inhibited the generation of IgE in serum and bronchoalveolar lavage fluid, and suppressed the expression of Th2 cytokine in the lung. Therefore, the Wogonin of the present invention can be effectively used as a composition for the prevention, treatment, and improvement of asthma.

### Manufacturing Example 1

### Preparation of Pharmaceutical Formulations

### <1-1> Preparation of Powders

| | |
|---|---|
| Wogonin | 500 mg |
| Lactose | 100 mg |
| Talc | 10 mg |

Powders were prepared by mixing all the above components, which were filled in airtight packs according to the conventional method for preparing powders.

### <1-2> Preparation of Tablets

| | |
|---|---|
| Wogonin | 500 mg |
| Corn starch | 100 mg |
| Lactose | 100 mg |
| Magnesium stearate | 2 mg |

Tablets were prepared by mixing all the above components by the conventional method for preparing tablets.

### <1-3> Preparation of Capsules

| | |
|---|---|
| Wogonin | 500 mg |
| Corn starch | 100 mg |
| Lactose | 100 mg |
| Magnesium stearate | 2 mg |

Capsules were prepared by mixing all the above components, which were filled in gelatin capsules according to the conventional method for preparing capsules.

### <1-4> Preparation of Injectable Solutions

| | |
|---|---|
| Wogonin | 500 mg |
| Injectable D.W | proper amount |
| pH regulator | proper amount |

Injectable solutions were prepared by mixing all the above components, which were filled in ampoules (2 ml/ampoule) according to the conventional method for preparing injectable solution.

### <1-5> Preparation of Liquid Formulations

| | | |
|---|---|---|
| Wogonin | 100 | mg |
| Isomerized sugar | 10 | g |
| Mannitol | 5 | g |

| | |
|---|---|
| Purified water | proper |
| | amount |

All the above components were dissolved in purified water. After adding lemon flavor, total volume was adjusted to be 100 ml by adding purified water. Liquid formulations were prepared by putting the mixture into brown bottles and sterilizing thereof by the conventional method for preparing liquid formulations.

### Manufacturing Example 2

### Preparation of Health Food

| Wogonin | 1000 | mg |
|---|---|---|
| Vitamin complex | proper | |
| | amount | |
| Vitamin A acetate | 70 | μg |
| Vitamin E | 1.0 | mg |
| Vitamin B1 | 0.13 | mg |
| Vitamin B2 | 0.15 | mg |
| Vitamin B6 | 0.5 | mg |
| Vitamin B12 | 0.2 | μg |
| Vitamin C | 10 | mg |
| Biotin | 10 | μg |
| Nicotinic acid amide | 1.7 | mg |
| Folic acid | 50 | μg |
| Calcium pantothenate | 0.5 | mg |
| Minerals | proper | |
| | amount | |
| Ferrous sulfate | 1.75 | mg |
| Zinc oxide | 0.82 | mg |
| Magnesium carbonate | 25.3 | mg |
| Potassium phosphate monobasic | 15 | mg |
| Potassium phosphate dibasic | 55 | mg |
| Potassium citrate | 90 | mg |
| Calcium carbonate | 100 | mg |
| Magnesium chloride | 24.8 | mg |

Vitamins and minerals were mixed according to the preferable composition rate for health food. However, the composition rate can be adjusted. The constituents were mixed according to the conventional method for preparing health food (ex. granules, etc) and then the composition for health food was prepared according to the conventional method.

### Manufacturing Example 3

### Preparation of Health Beverages

| | | |
|---|---|---|
| Wogonin | 1000 | mg |
| Citric acid | 1000 | mg |
| Oligosaccharide | 100 | g |
| Maesil (Prunus mume) Extract | 2 | g |
| Taurine | 1 | g |
| Purified water | up to 900 | ml |

The above constituents were mixed according to the conventional method for preparing health beverages. The mixture was heated at 85° C. for 1 hour with stirring and then filtered. The filtrate was loaded in 2 l sterilized containers, which were sealed and sterilized again, stored in a refrigerator until they would be used for the preparation of a composition for health beverages.

The constituents appropriate for favorite beverages were mixed according to the preferred mixing ratio but the composition ratio can be adjusted according to consumer characteristics, purpose of use, regional and national preferences, etc.

### Manufacturing Example 4

### Preparation of Other Health Foods

### <4-1> Preparation of Beverages

| | | |
|---|---|---|
| Honey | 522 | mg |
| Thioctic acid amide | 5 | mg |
| Nicotinic acid amide | 10 | mg |
| Hydrochloric acid riboflavin natrium | 3 | mg |
| Hydrochloric acid pyridoxine | 2 | mg |
| Inositol | 30 | mg |
| Ortho acid | 50 | mg |
| Wogonin | 0.48~1.28 | mg |
| Water | 200 | ml |

Beverages were prepared based on the above compositions and contents according to the conventional method.

### <4-2> Preparation of Chewing Gums

| | |
|---|---|
| Gum base | 20% |
| Sugar | 76.36~76.76% |
| Wogonin | 0.24~0.64% |
| Fruit flavor | 1% |
| Water | 2% |

Chewing gums were prepared based on the above compositions and contents according to the conventional method.

### <4-3> Preparation of Candies

| | |
|---|---|
| Sugar | 50~60% |
| Starch syrup | 39.26~49.66% |
| Wogonin | 0.24~0.64% |
| Orange flavor | 0.1% |

Candies were prepared based on the above compositions and contents according to the conventional method.

### <4-4> Preparation of Flour Food

0.5˜5.0 weight part of Wogonin was added to the flour. Health enhancing foods such as bread, cake, cookies, crackers and noodles were prepared with the flour mixture according to the conventional method.

### <4-5> Preparation of Dairy Products

5˜10 weight part of Wogonin was added to milk. Health enhancing dairy products such as butter and ice cream were prepared with the milk mixture according to the conventional method.

### <4-6> Preparation of Sun-Sik

Brown rice, barley, glutinous rice and Yulmu (Job's tears) were gelatinized according to the conventional method, dried and pulverized to obtain 60-mesh powders.

Black soybean, black sesame and wild sesame were steamed and dried according to the conventional method and pulverized to obtain 60-mesh powders.

The Wogonin of the present invention was concentrated under reduced pressure, spray-dried and pulverized to obtain 60-mesh dry powders.

Sun-Sik was prepared by mixing the dry powders of the grains, seeds and the Wogonin of the present invention according to the below ratio.

Grains (brown rice: 30 weight part, Yulmu: 15 weight part, barley: 20 weight part),

Seeds (wild sesame: 7 weight part, black soybean: 8 weight part, black sesame: 7 weight part),

Dry powders of the compound isolated from the extract of Example <3-2> (3 weight part),

(0.5 weight part),

(0.5 weight part)

| | |
|---|---|
| Brown rice | 30% |
| Yulmu | 15% |
| Barley | 20% |
| Wild sesame | 7% |
| Black soybean | 7% |
| Black sesame | 7% |
| Wogonin | 3% |
| Ganoderma lucidum | 0.5% |
| Rehmannia glutinosa | 0.5% |

Those skilled in the art will appreciate that the conceptions and specific embodiments disclosed in the foregoing description may be readily utilized as a basis for modifying or designing other embodiments for carrying out the same purposes of the present invention. Those skilled in the art will also appreciate that such equivalent embodiments do not depart from the spirit and scope of the invention as set forth in the appended Claims.

1-10. (canceled)

11. A method for treating asthma comprising administering to a subject in need thereof a pharmaceutically effective amount of Wogonin represented by the following formula 1, 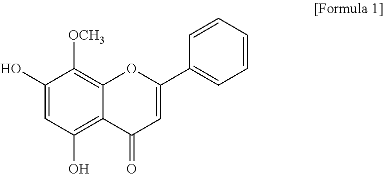 [SMILES: COc1c(O)cc(O)c2c(=O)cc(-c3ccccc3)oc12]
or a pharmaceutically acceptable salt thereof.

12. The method of claim 11, wherein the asthma is selected from the group consisting of bronchial asthma, allergic asthma, atopic asthma, non-atopic asthma, excise-induced asthma, aspirin-induced asthma, cardiac asthma, and alveolar asthma.

13. The method of claim 11, wherein treating comprises one or more of alleviating airway hypersensitivity, inhibiting reactive oxygen generation in the airway, and suppressing inflammatory cell infiltration in the bronchus.

14. The method of claim 11, wherein treating comprises one or more of reducing the number of inflammatory cells in the bronchus, reducing the concentration of IgE antibody in serum, and suppressing the expressions of Th2 cytokines.

15. A method for alleviating airway hypersensitivity or inhibiting reactive oxygen generation in the airway comprising administering to a subject in need thereof a pharmaceutically effective amount of Wogonin represented by the following formula 1, 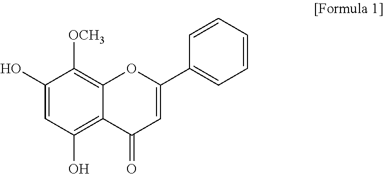 [SMILES: COc1c(O)cc(O)c2c(=O)cc(-c3ccccc3)oc12]
or a pharmaceutically acceptable salt thereof.

16. A method for reducing the number of inflammatory cells in the bronchus, the concentration of IgE antibody in serum, or the expression of Th2 cytokine comprising administering to a subject in need thereof a pharmaceutically effective amount of Wogonin represented by the following formula 1, 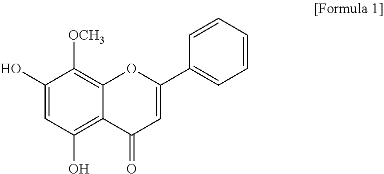 [SMILES: COc1c(O)cc(O)c2c(=O)cc(-c3ccccc3)oc12]
or a pharmaceutically acceptable salt thereof.
